(19) United States
(12) Patent Application Publication
(10) Pub. No.: US 2007/0141584 A1
(43) Pub. Date: Jun. 21, 2007

(54) Methods for assessment of native chromatin on microarrays

(76) Inventors: Douglas N. Roberts, Campbell, CA (US); Douglas A. Amorese, Los Altos, CA (US); Stephanie Fulmer-Smentek, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/313,522

(22) Filed: Dec. 20, 2005

(51) Int. Cl. C12Q 1/68; C12P 19/34

(52) U.S. Cl. 435/6; 435/91.2

(57) ABSTRACT

A method for determining chromatin accessibility of nucleic acids in a cell by expressing an effective amount of a nuclease in a cell to digest chromatin at chromatin accessible sites to form chromatin fragments; isolating chromatin fragments from the cell; and hybridizing the chromatin fragments on a microarray to determine the location and/or sequence of the chromatin fragments.

### BACKGROUND

DNA Microarray Technology is commonly used to determine the amounts of a given species of nucleic acid in a sample relative to a reference sample. In array based comparative genomic hybridization (aCGH), genomic DNA is purified away from cellular components of reference and test cells to determine differences in genomic copy number. The purified genomic DNA from reference and test cells is differentially labeled and then hybridized competitively to a microarray containing probes representing the genome. Chromosomal regions in the test cells bearing an altered copy number from that of the reference cells can be identified based upon differential hybridization signals. However, the isolation of genomic DNA for CGH destroys protein-DNA interactions. All of the protein content, including special and higher order chromatin organization is lost during purification of the DNA. Thus, the information obtained in a hybridization is limited to only differences in copy number.

Chromatin immunoprecipitation (ChIP) utilizes chromatin from cells that have been treated with cross-linking agents. The cross-linked chromatin is subsequently sheared into approximately 1 kilobase fragments. Specific segments of this chromatin are then isolated from the sample using antibodies directed against proteins that have been cross-linked to the DNA. The isolated DNA is then labeled and hybridized to a genomic microarray. The starting or input DNA for the immunoprecipitation is labeled and used as a reference standard in a competitive hybridization. Segments of DNA that are enriched by the immunoprecipitation will have a higher signal on the array than those that are not enriched. ChIP allows determination of protein DNA-binding events. However, cross-linking, lysis and mechanical disruption of the chromatin may introduce biases in the recovery of chromatin both before and after an immunoselection. In addition, ChIP focuses on individual protein-DNA binding events, providing little information on chromosomal positioning, domain, or higher order DNA structure.

In these and other techniques to analyze chromatin structure, the living state of the cell is disrupted and destroyed. Information gained is limited to linear relationship to the genomic sequence. Consequently, there is a need in the art for improved methods for identifying the native state of chromatin, particularly as related to regulation of gene expression and positional, three-dimensional relationship within native chromatin.

### SUMMARY

A method for determining chromatin accessibility of nucleic acids in a cell by expressing an effective amount of a nuclease in a cell to digest chromatin at chromatin accessible sites to form chromatin fragments; isolating chromatin fragments from the cell; and hybridizing the chromatin fragments on a microarray to determine the location and/or sequence of the chromatin fragments.

In the inventive method, the expressed nuclease includes one or more of deoxyribonuclease, micrococcal nuclease, and/or restriction endonucleases. The methods of the present invention may optionally be combined with other techniques such as cross-linking, immunoselection, and size fractionation.

### BRIEF DESCRIPTION OF THE DRAWINGS

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

### DETAILED DESCRIPTION

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

### DEFINITIONS

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

The term “chromatin” refers to the structure comprised of DNA and proteins that a eukarotic genome is packed into. The structural unit of chromatin is an assemblage, called the nucleosome, composed of five types of histones (designated H1, H2A, H2B, H3, and H4) and approximately 1.8 turns of DNA wound around a core particle of the histone proteins. Approximately 166 base pairs are bound to the nucleosome: 146(±1) base pairs are tightly bound to the core particle and the remaining 20 base pairs are associated with the H1 histone. This nucleosome structure is conserved in eukaryotes. In native environment, the majority of the chromatin is present in higher-order chromatin fibers about 25 to 35 nm in diameter, which may be further organized into looped domains.

The region of DNA between two nucleosomes is variable in length and is referred to as a linker segment. Chromatin repeat length, which is the linker length plus DNA base pairs bound to the nucleosome, usually increases with increases in transcriptional activity. Regulatory proteins bind to DNA and cause local nucleosome phasing or realignment of the nucleosomes at regular intervals along the chromosome. Because nucleosome phasing is caused by the binding of regulatory proteins to the DNA, it is typically limited to non-coding regions involved in gene regulation. The DNA segments free of nucleosomes, also called chromatin accessible sites, are exposed to chemical and enzymatic attack. The presence of accessible sites is generally correlated with gene activity.

The term “locus” refers to a fixed position in a genome corresponding to a gene. A locus may have an associated “locus control region” which refers to a segment of DNA that controls the chromatin structure and thus the potential for replication and transcription of an entire gene cluster.

The term “nuclease” refers to any of several enzymes that hydrolize nucleic acids. Nucleases may be non-specific, specific for types of nucleic acid such as DNA or RNA, and/or specific for single or double stranded forms of nucleic acids. Nucleases include various overlapping categories of enzymes, for example, deoxynucleases, which specifically hydrolize DNA, and endonucleases which are nucleases that cleave nucleic acids at interior bonds and so produce fragments of various sizes.

The term “genome” refers to all nucleic acid sequences (coding and non-coding) and elements present in or originating from a single cell or each cell type in an organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, eukaryotic genomes in their native state have regions of chromosomes protected from nuclease action by higher order DNA folding, protein binding, or subnuclear localization. The method of the present invention can be used to identify either these protected regions or the unprotected regions in a genome-wide (high throughput) fashion.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins) or polysaccharides (starches, or polysugars), as well as other chemical entities that contain repeating units of like chemical structure.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “functionalization” as used herein relates to modification of a solid substrate to provide a plurality of functional groups on the substrate surface. By a “functionalized surface” is meant a substrate surface that has been modified so that a plurality of functional groups is present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “oligonucleotide bound to a surface of a solid support” refers to an oligonucleotide or mimetic thereof, e.g., peptide nucleic acid or PNA, that is immobilized on a surface of a solid substrate in a feature or spot, where the substrate can have a variety of configurations, e.g., a sheet, bead, or other structure. In certain embodiments, the collections of features of oligonucleotides employed herein are present on a surface of the same planar support, e.g., in the form of an array.

The term “array” encompasses the term “microarray” and refers to an ordered array presented for binding to nucleic acids and the like. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus).

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there are intervening areas which lack features of interest.

An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic, flexible web and other materials are also suitable.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The term “sensitivity” refers to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

### METHODS OF THE PRESENT INVENTION

The present invention provides methods for isolating chromatin from cells so as to capture its native state with respect to structure, localization, and protein binding, and comparing populations of nucleic acids, one example being array Comparative Genomic Hybridization (aCGH) applications.

The subject invention is directed to methods of analyzing native chromatin using an array of substrate immobilized oligonucleotide features. The subject invention includes methods for isolating chromatin from cells so as to capture its native state with respect to structure, localization, and protein binding. One embodiment of the inventive method includes expression of a nuclease in a living cell. The expression of the nuclease provides selection for or against accessible regions of chromatin in living cells. In some embodiments, the nuclease is under the control of an inducible promoter. The DNA is then compared to differentially labeled reference DNA on an array. The methods of the present invention may optionally be combined with other techniques such as cross-linking, immunoselection, size fractionation, and chromatin solubility assay techniques.

The subject invention provides methods for isolating chromatin from cells so as to capture its native state with respect to structure, localization, and protein binding, and subsequently compare populations of nucleic acids to determine regions of chromatin accessiblity.

In one aspect of the invention, a method to provide one or more populations or collections of nucleic acids/chromatin that are to be compared to a standard, control, or each other. The two or more (i.e., at least first and second, where the number of different collections may, in certain embodiments, be three, four or more) populations of nucleic acids are prepared from different populations of cells. The cells may be obtained from any type of tisse, may be obtained from diseased tissue, may have been treated with one or more agents or drugs, may be cell lines, and/or may be stem cells. These are eukarotic cells that have nucleic acids structured as chromatin. As such, the first step in many embodiments of the subject methods is to prepare a collection of nucleic acids from chromatin, from an initial genomic source for each cell or population thereof that is to be compared.

In the subject invention, a cell includes an expression system for at least one nuclease, wherein expression of the nuclease in the cell results in the digestion of chromatin at accessible sites. In an embodiment, the cell includes an expression system for the nuclease without any major disturbance to the living cell.

The method of the subject invention provides methods for intracellular generation of chromatin fragments wherein the fragments generated closely reflect or capture native state of the chromatin in the cells. The methods for generating fragments is applied to one or more different populations of cells wherein with respect to structure, localization, and protein binding, and subsequently probing genomic arrays and/or against other populations of nucleic acids.

Chromatin fragments are generated by intracellular expression of nuclease in the living cells. In an embodiment, the expression of the nuclease and time for activity of the nuclease in the cell can be manipulated. For example, a nuclease expression system can be designed to express nuclease shortly after introduction of an inducing agent or withdrawal of an inhibitor or silencing agent. The nuclease gene is expressed in the cells, transcribed into protein, transported in the nucleus, and acts upon accessible sites of the chromatin. In an embodiment, the nuclease is allowed to act for an extended period of time. In an alternative embodiment, after a period of time for action of the nuclease in the cells, the nuclease activity is halted by methods such as change in temperature, introduction of nuclease inhibitors such as antibodies there to or nuclease specific cleavage agents, introduction of cross-linking agents, or cell lysis followed by protein denaturation.

### Nucleases

The nuclease is actively expressed in the cells for action on the chromatin located in the cell nucleus. The chromatin is digested into chromatin fragments within the cell. The nuclease is preferably a deoxyribonuclease. The nuclease may be either endogeneous or heterologous to the cells. In one embodiment, a endogenous nuclease, such as a native DNAse, is induced to greater than normal expression and/or activity levels in the cells. In another embodiment, a heterologous nuclease is expressed within the cells by various known cloning and expression methods. Nuclease digestion results in chromatin fragments within the cell.

The type and amount of nuclease produced, as well as time of action will affect the number of cuts made in turn affecting the size and quantity of chromatin fragments generated. The sequence specificity, processivity, and amount expressed of the nuclease, in combination with a time period for digestion are selected based on the amount of digestion required to generate the desired population of chromatin fragments for analysis. A time period for digestion begins from expression of the nuclease until stopped or digestion is complete. A single time period for digestion may be employed. Alternatively, multiple time periods for digestion, wherein a population of cells (chromatin fragments) is collected at two or more time points during the digestion time period. Each population of cells is collected and digestion stopped. Digestion may be stopped by any of a number of known methods, including for example but not limited to, rapid change in temperature (e.g., heat or freezing), treatment with nuclease inhibitors, physical separation of nuclease protein from chromatin, and cross-linking.

In some embodiments, the chromatin is cut a limited number of times, for example by using a sequence specific nuclease and/or limiting the time period for action of either a specific or non-specific nuclease. In an embodiment, the chromatin is cut as little as once. In some embodiments, a limited time period is two hours or less, measured from induction or beginning of nuclease expression. In further embodiments, a limited time period is one hour or less, 45 minutes or less, or 30 minutes or less. In embodiments, where the nuclease makes few cuts to the chromatin, large segments of DNA representing individual nucleosomes and/or higher order loops of entire chromosomal regions are generated or liberated.

In other embodiments, many cuts are made to the chromatin to generate or liberate larger numbers of chromatin fragments. In various embodiments, higher levels of digestion are achieved by longer time periods for digestion and/or expression of non-sequence specific nucleases such as DNAse I or micrococcal nuclease (MNase). Digestion with non-sequence specific nucleases over longer time periods, for example, several hours to days, cut the accessible DNA into very small fragments or degrade the nuclease-accessible DNA, such that the remaining chromatin fragments represents the most protected chromosome segments in the cell.

Examples of sequence specific nucleases include restriction endonucleases, which cut only at sequence defined locations on the DNA. The number of cuts for sequence specific nucleases is limited by the occurance and accessibility of the target sequence. The most commonly used restriction endonucleases cut at a specific four or six base pattern generating either blunt or overhanging ends. The cutting patterns, genes and source organisms of the various restriction enzymes are known and available. Information regarding a variety of restriction endonucleases is available from companies such as New England Biolabs® Inc., Ipswich, Mass. and Promega Corporation, Madison, Wis. For example, the restriction enzyme, Bam HI is produced by the BamH I gene from Bacillus amyloliquefaciens H (ATCC 49763).

The average DNA size of the chromatin fragments following nuclease-digestion chromatin depends on the type and amount of nuclease produced, and time of action within the cell, as described above. In certain embodiments, the chromatin fragments typically have an average size of at least about 1 Mb. In other embodiments, the chromatin fragments have an average size of less than about 1 Mb. In some embodiment, the chromatin fragments have a representative range of sizes from about 50 to about 250 Mb or more. In still other embodiments, the sizes may not exceed about 50 MB. In further embodiments, they may be about 1 Mb or smaller, e.g., less than about 500 Kb, etc. In many embodiments, the chromatin fragments following digestion include both large chromatin fragments (e.g., greater than 1 Mb) and smaller chromatin fragments.

### Cells

Chromatin for analysis by the present methods is found in cells. Cells are a population of eukaryotic cells which act as a genomic source. Eukaryotes include mammals, plants, and fungi. Example organisms include, but are not limited to human, monkey, cow, horse, dog, cat, rat, mouse, chicken, alligator, frog, carp, silkworm, fruit fly, flatworm, freshwater hydra, nematode, yeast, green algae, barley, wheat, corn, tomato, tobacco, pine, garden pea, rice, potato, and broad bean. Other examples of sources include animal cells, plant cells, virus-infected cells, immortalized cell lines, cultured primary tissues such as mouse or Human fibroblasts, stem cells, embryonic cells, diseased cells such as cancerous cells, transformed or untransformed cells, fresh primary tissues such as mouse fetal liver, or extracts or combinations thereof.

Cells may be prepared from a subject, for example a plant or an animal, virus-infected cells, immortalized cell lines, cultured primary tissues such as mouse or Human fibroblasts, stem cells, embryonic cells, diseased cells such as cancerous cells, transformed or untransformed cells, fresh primary tissues such as mouse fetal liver, or extracts or combinations thereof. In certain embodiments, the genomic source is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse genomic sources. In certain embodiments, a set of nucleic acid sequences within the genomic source is complex, as the genome contains at least about 1×10⁸ base pairs, including at least about 1×10⁹ base pairs, e.g., about 3×10⁹ base pairs.

The methods of the present invention for analysis of chromatin are suitable for any eukaryotic cell. The methods are applicable to both dividing cells and non-dividing cells. In various method embodiments, cells in a particular cell cycle stage, i.e., G₀, G₁, G₂, G₃, G₄, are targeted for analysis. The chromosomes of dividing cells, for example somatic cells in mitosis and gametes in meiosis, are condensed. Condensed metaphase chromosomes from dividing cells may be analyzed by the methods described herein by activating nuclease expression when cells are actively dividing. Other techniques used in the art for aligning cell cycle stage of a cell population may also be combined with the present method.

### Endogeneous Expression

In various embodiments, a endogenous nuclease, such as a native DNAse, is induced to greater than normal expression and/or activity levels in the cells. In one embodiment, for example, additionally regulatory sequences are introduced into the locus for human DNaseI. The additional regulatory sequences bypass the native regulatory sequences to activate DNaseI expression. The additional regulatory sequences may be introduced by recombinant genomic cloning techniques. Various regulatory sequences and promoters are described below. In an alternative embodiment, DNaseI expression is induced by treatment of the cells with an agent, for example as described in DNase I mediates internucleosomal DNA degradation in human cells undergoing drug-induced apoptosis. Eur. J. Immunol. 31 (3), 743-751 (2001). In some embodiments, methods of the present invention may be used to analyze chromatin fragments liberated by drug-induced apoptosis.

### Heterologous Expression Systems

In another embodiment, a heterologous nuclease is introduced into the cells by various known cloning methods for expression.

In the present method, the nuclease is expressed in the living cells of interest. The description below relates to methods of producing nuclease by culturing cells transformed or transfected with a vector containing the encoding nucleic acid. (See, e.g., Sambrook et al., Molecular Cloning: A Laboratory Manual (New York: Cold Spring Harbor Laboratory Press, 1989). Transfection for purposes of the present invention may be either stable or transient.

In an embodiment, the nucleic acid (e.g., cDNA or genomic DNA) encoding the selected nuclease is inserted into a replicable vector for further cloning (amplification of the DNA) or for expression. DNA encoding a nuclease are available from the genome of the source organism. For example, the cloning of human DNAse I from a human source is described by Takeshita et al., (2001) Exp. Xlin. Immunogenet. 18:226-232. The mRNA coding sequence is given at gi:58331227. Bacterial strains and other microbial sources are available from facilities such as American Type Culture Collection (ATCC) of Manassass, Va. Sequence and source information is generally available from commercial enzyme suppliers, database sources such as GenBank and in the literature. For example: EcoRI has published sequence information at gi:152447 and Greene, P. J., Gupta, M., Boyer, H. W., Brown, W. E. and Rosenberg, J. M., Sequence analysis of the DNA encoding the Eco RI endonuclease and methylase (1981) J. Biol. Chem. 256 (5), 2143-2153.

Various vectors are publicly available. The vector components generally include, but are not limited to, one or more of the following: a signal sequence, an origin of replication, one or more marker genes, an enhancer element, a promoter, and a transcription termination sequence, each of which is described below. Targeting the expressed nuclease to the nucleus is achieved by appending a nuclear localization signal sequence to the nuclease gene.

Expression and cloning vectors usually contain a promoter that is recognized by the host organism and is operably linked to the encoding nucleic acid sequence. Promoters are untranslated sequences located upstream (5′) to the start codon of a structural gene (generally within about 100 to 1000 bp) that control the transcription and translation of a particular nucleic acid sequence, to which they are operably linked. Promoter sequences, inducible and constitutive, are known for eukaryotes. In various embodiments, nuclease expression is reversibly suppressed or silenced. Example means for suppression or silencing of nuclease expression include controlled expression of silencing RNA (siRNA) or promoter specific inhibitory proteins. siRNA products are commercially available from, for example, Ambion, Austin, Tex. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to the encoding DNA by removing the promoter from the source DNA by restriction enzyme digestion and inserting the isolated promoter sequence into the vector.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, glyceraldehyde-3-phosphate dehydrogenase, and enzymes responsible for maltose and galactose utilization.

Vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus (CMV), a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

One example expression system demonstrated to express DNAseI in mammalian cells is described by Takeshita et al. (2001) Exp. Clin. Immunogenet. 18:226-232, which includes a CMV promoter and pcDNA3.1 vector (Invitrogen, Carlsbad, Calif.). Another example mammalian expression system utilized recombinant adenovirus expressing Cre recombinase. A second recombinant adenovirus containing an on/off-switching reporter unit, where a gene (e.g., a nuclease) can be activated by the Cre-mediated excisional deletion of an interposed stuffer DNA. The two adenovirus contructs are coinfected into mammalian cells for expression of the gene of interest. Kanegae et al. (1995) Nucleic Acids Research 23:3816-3821.

A number of steroid-regulated promoters are also known for use in expression systems for plants and animals. See, for example, U.S. Pat. No. 5,512,483, EP 1232273 A2, EP 1242604 A2, U.S. Pat. No. 6,379,945, and EP 1,112360 A1 An expression system employing a dexamethasone (DM)-inducible promoter for mammalian expression is described by Klessig et al. (1984) Molecular and Cellular Biology, 4:1354-1362.

### Enhancer Elements

Transcription of a DNA by higher eukaryotes may be increased by inserting an enhancer sequence into the vector. Enhancers are cis-acting elements of DNA that act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, a-fetoprotein, and insulin) and eukaryotic cell viruses, such as the SV40 enhancer, the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters.

### Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs.

### Construction and Analysis of Vectors

Construction of suitable vectors containing one or more of the above-listed components employs standard ligation techniques. Isolated plasmids or DNA fragments are cleaved, tailored, and re-ligated in the form desired to generate the plasmids required. Expression vectors that provide for the transient expression of nuclease in mammalian cells of the encoding DNA may be employed. In general, transient expression involves the use of an expression vector that is able to replicate efficiently in a host cell, such that the host cell accumulates many copies of the expression vector and, in turn, synthesizes high levels of the nuclease encoded by the expression vector [Sambrook et al., supra].

Cells are transfected and preferably transformed with the above-described expression or cloning vectors and cultured in nutrient media modified as appropriate for control of nuclease expression and cell growth or survival.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. Infection with Agrobacterium tumefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, may also be used. For various techniques for transforming mammalian cells, see Keown et al., Methods in Enzymology, 185:527-537 (1990) and Mansour et al., Nature, 336:348-352 (1988).

Mammalian and yeast cells can be cultured in suitable culture media as described generally in Sambrook et al., supra. Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

### Isolation and Manipulation of Chromatin Fragments

According to the methods described above, chromatin is intracellularly digested thereby generating chromatin fragments. Chromatin fragments refers to DNA fragments with or without associated proteins. Chromatin fragments corresponding to nucleosome-bound or protein-bound regions may be recovered from the cells with associated proteins. Proteins associated with chromatin fragments may be sequence-specific or sequence non-specific DNA binding factors, including, but are not limited to, transcriptional regulatory proteins (e.g., activators and repressors), enzymes (e.g.,nucleases, RNA polymerases), and histones and other housekeeping proteins.

In various embodiments, chromatin fragments from a control or reference population of cells is generated by methods of the present invention or according to conventional techniques for comparison with chromatin fragments generated according to the present invention. In one embodiment, chromatin fragments are generated from two or more populations of cells, wherein at least one population of cells serves as a reference or control.

Isolation of chromatin fragments from a population of cells may be accomplished by a number of techniques. The liberated chromatin fragments in the cells may be subsequently prepared for analysis using any convenient protocol. In many embodiments, the cells are converted into a cell lysate. A chromatin fragment-containing fraction of the cell lysate is subsequently obtained by any convenient means and numerous protocols for doing so are well known in the art. Typically, the liberated chromatin fragments are soluble or may be solubilized for separation from insoluable cellular debris. In some embodiments, non-denaturating conditions are employed. Further purification techniques are optionally applied to the chromatin fragments dependent on the information desired from array hybridization.

Additional optional isolation and manipulation steps are described below. The additional steps are performed after nuclease digestion, but may be performed before or after recovery of chromatin fragments from the cells, as appropriate for each technique.

In certain embodiments, an optional cross-linking step is applied to the cells after nuclease activation. Preferably, cross-linking is performed before recovery of the chromatin fragments from the cells. The cells are contacted with cross-linking agents to cross-link the chromatin (e.g., chemically link any DNA-bound proteins to the DNA). Examples of common cross-linking agents include formaldehyde and EDC (ethyldimethylaminopropylcarbdiimide). One basic method for cross-linking involves permeabilizing the cell membranes and introducing the cross-linking agents into a solution containing the permeabilized cells. Various cross-linking methods, including, but not limited to DNA-protein and protein-protein cross-linking, are known in the art.

In an embodiment, the chromatin fragment-containing fraction, particularly where, but not limited to embodiments with cross-linking after nuclease digestion, is selectively enriched by immunoprecipitation. Immunoprecipitation techniques on chromatin, such as ChIP are known in the art. See, Sambrook supra.

In other embodiments, typically those without cross-linking steps, the chromatin fragments are optionally separated from associated proteins. These purification steps are also known in the art. See, Sambrook supra. The isolated chromatin fragments may optionally undergo further optional purification such as RNA digestion and size fractionation. Size fractionation of chromatin fragments may be used, for example, to separate larger fragments from the smaller fragments. As described above, size of chromatin fragments may be used to differentiate protected regions of DNA from hypersensitive or linker regions.

In some embodiments, the chromatin fragments are isolated and regions flanking the accessible sites may be optionally amplified. The fragments may be optionally sub-cloned into a suitable vector, such as a commercially available bacterial plasmid, or amplified by PCR.

### Labeling of Chromatin Fragments

Prior to or during analysis, the populations of chromatin fragments are typically labeled. The populations may be labeled with the same label or different labels, depending on the actual assay protocol employed. For example, where each population is to be contacted with different but identical arrays, the DNA from each population or collection may be labeled with the same label. Alternatively, where both populations are to be simultaneously contacted with a single array of immobilized oligonucleotide features, i.e., cohybridized to the same array of immobilized nucleic acid feature, solution-phase collections or populations of the DNA that are to be compared are generally distinguishably or differentially labeled with respect to each other.

In an embodiment, chromatin fragments are detected by fluorescence measurements by labeling with a fluorescent dye or other marker sufficient for detection through an automated DNA microarray reader. The labeled fragment population generally is incubated with the surface of the DNA microarray onto which has been spotted different binding moieties and the signal intensity at each array coordinate is recorded. Fluorescent dyes such as Cy3 and Cy5 are particularly useful for detection.

In some embodiments, the chromatin fragments are not labeled, in accordance with the particular detection protocol employed in a given assay. For example, in certain embodiments, binding events on the surface of a substrate may be detected by means other than by detection of labeled nucleic acids, such as by change in conformation of a conformationally labeled immobilized oligonucleotide, detection of electrical signals caused by binding events on the substrate surface, etc.

### Arrays

As indicated above, the arrays are arrays of nucleic acids, including oligonucleotides, polynucleotides, DNAs, RNAs, synthetic mimetics thereof, and the like. The subject arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

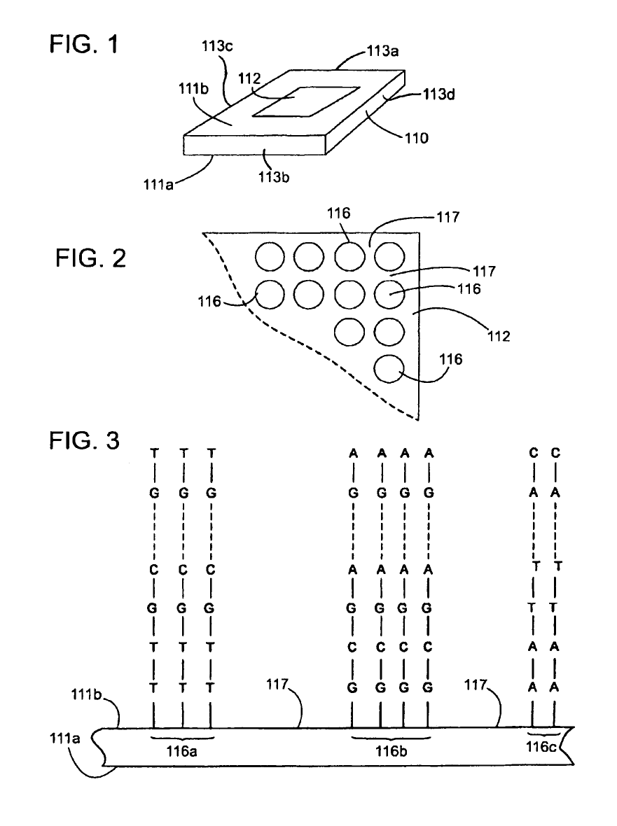

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

### Methods of Profiling on Arrays

In an embodiment, the nuclease-treated chromatin generated from the cells is used as a probe to hybridize against a population of nucleic acid sequences on a microarray. In one embodiment, those sequences correspond to set of previously characterized linker regions or hypersensitive regions in a genome. In another embodiment, those sequences form a tiled array physically spanning a section to totality of a genome. In one embodiment, those sequences correspond to large combination of oligonucleotides for determination of complex binding patterns. Following analysis the presence and intensity of the signal from spots on the array reflects the nature of that nuclease-sensitive or nuclease-protected site within that population of cells.

In one embodiment, two or more arrays are prepared under similar conditions with one array acting as a control or reference for the other(s). For example, alteration of expression induced by a test compound such as a drug candidate may be determined by creating two arrays, one that corresponds to cells that have been treated with the test compound and a second that corresponds to the cells before treatment.

In another embodiment, array data is used to identify structure and function of regions of genomic DNA. Hypersensitive and linker regions in native chromatin may be identified. Genomic DNA regions coordinated with nucleosomes may be identified. Furthermore, various cell states or environmental effects on chromatin in vivo, for example growth conditions, cell cycle state, gene activation, exposure to chemicals or other stimuli are readily assayed by the subject methods.

Differences in array data profiles can reveal which chromatin fragments are affected by a test compound administered to the cells. A chromatin fragment may be more hypersensitive in the presence of the compound, as seen by more nuclease digestion leading to a stronger chromatin fragment signal in an array profile as compared to no compound control cells. A chromatin fragment may be found less hypersensitive if, in comparison to a no compound control, a weaker signal was produced for that chromatin fragment spot in the array.

In another embodiment, an array profile obtained from a malignant tissue sample may be compared with an array profile obtained from a control or normal tissue sample. An inspection of the hypersensitive chromatin fragment differences between the arrays may reveal a genetic cause in the disease or a genetic factor in the disease progression.

In another embodiment, an array generates data that reveals fragment copy number. As will be readily appreciated, some chromatin fragments are more hypersensitive than others for a given cell state and this character can be seen as a higher copy number, or (where appropriate) a greater detection signal compared to another chromatin fragment or reference sample. According to an embodiment of the invention, the relative copy numbers of one or more chromatin fragments are compared to a reference or set of references to determine a relative activity of the DNA fragment.

The subject array methods find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In various embodiments, the array hybridization conditions are controlled for specific or selective hybridization of chromatin fragments, including nucleic acid probes derived therefrom, to the array. Specific or selective hybridization refers to the binding, duplexing, or hybridizing of a nucleic acid molecule of a chromatin fragment preferentially to a particular nucleotide sequence on the array under stringent conditions.

Stringent assay conditions as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A stringent hybridization and stringent hybridization wash conditions in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

In using an array, the array will typically be exposed to a sample (for example, a fluorescently labeled analyte, e.g., chromatin fragment including a labeled DNA) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. patent applications: Ser. No. 09/846125 “Reading Multi-Featured Arrays” by Dorsel et al.; and Ser. No. 09/430214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### PROPHETIC EXAMPLE

Illustrative Method for the Production of Chromatin Fragments for use in Hybridization to Microarrays

### A. Preparation of Chromatin Fragments

A DNA fragment containing the entire coding sequence of human DNaseI is cloned into a mammalian expression vector, pMDSG (AC IG0091; Amersham) under the control of a dexamethasone (DM)-inducible promoter from MMTV. HeLa cells are transfected with supercoiled or EcoRI linearized vector and selected for growth in the presence of mycophenolic acid. A line of HeLa cells carrying the DNase I gene is treated with DM to induce DNase I expression. The HeLa cells are maintained in favorable growth conditions for an additional 1 to 24 hours to generate chromatin fragments in the cells. After the specified digestion period, for example 1 hour, the cells will undergo one or more further processing steps described below. The reaction of DNase I with the chromatin is stopped by adding EDTA to approximately 10 mM and chilling the cells on ice.

The Chromatin fragments may be fractionated by ultracentrifugation in sucrose gradients. The cells are lysed by either physical or chemical means and layered onto a 5-30% sucrose gradient and spun 16 hours at 28000 rpm. The size of the DNA fragments is determined by agarose gell electophoresis or other suitable methods. In one embodiment, subnucleosomal size (e.g., less than 150 bp) are labled for use as probes. In an alternative embodiment, after fractionation, the fractions are treated with 50 μg/mL RNase for 30 minutes at 37° C., followed by treatment with EDTA, SDS and Proteinase K. The fractions are then phenol-chloroform extracted and ethanol precipitated for DNA recovery.

The recovered DNA probes are labeled with Cy3 or Cy5 by suspending in water or buffer, and adding a solution of random primers, such as is available in Invitrogen's BioPrime Labeling Kit. A mixture of 5 mM dNTP solution is added with 1 mM dCtp-Cy3 or 1 mM dCTP-Cy5 and Klenow. The mixture is incubated for 2.5 hours at 37° C. before stopping by addition of EDTA. The probes are purified, for example by Qiagen's QIAquick column. The amount of incorporation is calculated by reading the absorbance at 550 nm for Cy3 and 650 nm for Cy5.

### B. Optional Crosslinking of DNA to Protein within Chromatin Fragments

Start with cells that have undergone internal nuclease digested according to the above methods. Centrifuge to pellet the cells or nuclei, wash and resuspend in buffer, such as PDS pH 7.4 with 1 mM EDTA and 0.5 mM EGTA and freshly added protease inhibitors. Add formaldehyde to a final concentration of 0.5% and mix gently at room temperature for 10-15 min. Quench crosslinking reaction by adding 2.5 M glycine to a final concentration of 125 mM. Stir at room temperature for an additional 5 min. Pellet cells or nuclei by centrifugation and resuspend in buffer. Lyse cells and/or nuclei and recover the DNA-protein complexes.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for determining chromatin accessibility of nucleic acids in a cell comprising:
a) expressing an effective amount of a nuclease in a cell, wherein amount of the nuclease is sufficient to digest chromatin at chromatin accessible sites to form chromatin fragments;
b) isolating chromatin fragments from the cell; and hybridizing the chromatin fragments on a microarray to determine the location and/or sequence of the chromatin fragments.

2. The method of claim 1, wherein determining the location and/or sequence of the chromatin fragments comprises comparing the hybridization profile of the chromatin fragments from the cell to the hybridization profile of chromatin fragments from a control cell not subjected to the expressed nuclease and identifying the location and/or sequence of the chromatin fragments as those locations and sequences that are different from the control.

3. The method of claim 1, wherein hybridizing the chromatin fragments on a microarray determines the location and/or sequence of the chromatin accessible sites.

4. The method of claim 1, wherein hybridizing the chromatin fragments on a microarray determines the location and/or sequence of the sequestered sites.

5. The method of claim 1, wherein the nuclease is under the control of an inducible promoter.

6. The method of claim 1, further comprising introducing a nucleic acid encoding a nuclease under the control of an inducible promoter into the cell; and culturing the cell under conditions suitable for induction of expression of the nuclease.

7. The method of claim 1, wherein, the nuclease is DNase.

8. The method of claim 1, wherein, the nuclease is micrococcal nuclease.

9. The method of claim 1, wherein, the nuclease is a restriction endonuclease.

10. The method of claim 1, wherein the cell is a mammalian cell.

11. The method of claim 1, wherein the cell is a human cell.

12. The method of claim 1 further comprising treating the cells with cross-linking agents.

13. The method of claim 12 further comprising immunoprecipitating one or more chromatin fragments.

14. The method of claim 1, wherein the chromatin fragments are bound by one or more sequence-specific DNA binding factors.

15. The method of claim 1 further comprising size fractionating the chromatin fragments.

16. The method of claim 1, wherein the chromatin fragments are each a nucleotide sequence from a hypersensitive region or linker region.

17. The method of claim 1, wherein the microarray comprises immobilized oligonucleotide features.

18. The method of claim 1, wherein the microarray comprises a plurality of polynucleotides, each affixed to a substrate, the plurality comprising different polynucleotides differing in nucleotide sequence and being situated at distinct loci of the array, the different polynucleotides being complementary and hybridizable to genomic DNA of the chromatin fragments.
